(19) United States
(12) Patent Application Publication
(10) Pub. No.: US 2019/0232006 A1
(43) Pub. Date: Aug. 1, 2019

(54) ENDOTRACHEAL TUBE SYSTEM

(71) Applicants: HAMAD MEDICAL CORPORATION, Doha (QA); HAMAD MEDICAL CORPORATION GLOBAL PATENT TRUST, Manassas, VA (US)

(72) Inventor: Amr Salah Ibrahim Elsid OMAR, Doha (QA)

(73) Assignees: Hamad Medical Corporation, Doha (QA); Hamad Medical Corporation Global Patent Trust, Manassas, VA (US)

(21) Appl. No.: 16/316,031

(22) PCT Filed: Jan. 4, 2017
(86) PCT No.: PCT/US2017/012193

§ 371 Date: Jan. 7, 2019

Related U.S. Application Data
(60) Provisional application No. 62/359,944, filed on Jul. 8, 2016.

(51) Int. Cl. A61M 16/04; A61M 39/16

(52) U.S. Cl. CPC A61M 16/0434; A61M 16/0463; A61M 16/20; A61M 39/16; A61M 16/0479

(57) ABSTRACT

The endotracheal tube system (10) includes an endotracheal tube (12), an auxiliary sterilizing tube (20), and a sterilizing agent source (28) for sterilization of the endotracheal tube (12) while it is in use. The endotracheal tube (12) is an elongated, hollow tube having a distal end (14) and a proximal end (16). An inflatable cuff (18) is disposed on the elongated hollow tube (12), adjacent the distal end (14). The sterilizing tube (20) has a closed distal end (24) and an open proximal end (22). The closed distal end (24) is secured to the elongated hollow tube (12) adjacent the inflatable cuff (18), and a plurality of pores (26) are formed through at least a portion of the sterilizing tube (20). The sterilizing agent source (28) injects chlorhexidine into the sterilizing tube (20), which is distributed to the elongated hollow tube (12) through pores (26).

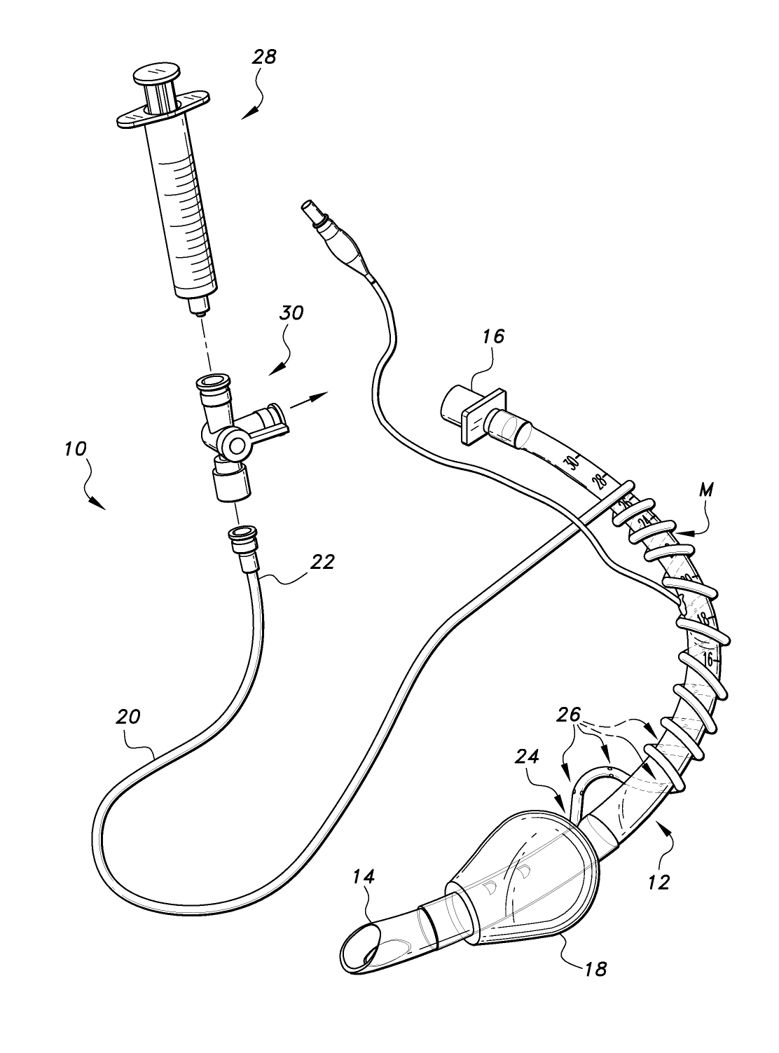

### TECHNICAL FIELD

The present invention relates to the sterilization of endotracheal tubes, and particularly to an endotracheal tube system including an endotracheal tube, an auxiliary sterilizing tube, and a sterilizing agent source, such as an infusion syringe or the like, for treating the endotracheal tube with a sterilizing agent.

### BACKGROUND ART

Ventilator-associated pneumonia (VAP) is a type of lung infection that occurs in people who are on breathing machines in hospitals. Prevention of VAP involves limiting exposure to resistant bacteria, discontinuing mechanical ventilation as soon as possible, and a variety of strategies to limit infection while intubated. Resistant bacteria are spread in much the same ways as any communicable disease. Proper hand washing, sterile technique for invasive procedures, and isolation of individuals with known resistant organisms are all mandatory for effective infection control. A variety of aggressive weaning protocols to limit the amount of time a person spends intubated have been proposed. Other recommendations for preventing VAP include raising the head of the bed to at least 30°, as well as utilization of endotracheal tubes with subglottic suction. Antiseptic mouthwashes, such as chlorhexidine, may also reduce the incidence of VAP. It would be desirable to be able to prevent VAP without requiring the patient to undergo additional procedures which might be stressful or harmful while the patient is being treated for his or her primary condition. Thus, an endotracheal tube with an auxiliary sterilizing tube solving the aforementioned problems is desired.

### DISCLOSURE OF INVENTION

The endotracheal tube system includes an endotracheal tube, an auxiliary sterilizing tube, and a sterilizing agent source, such as an infusion syringe or the like, for sterilization of the endotracheal tube while it is in use. The endotracheal tube can be an elongated hollow tube, such as a conventional endotracheal tube, having a distal end and a proximal end, with the distal end being adapted for insertion into a trachea of a patient. An inflatable cuff is disposed on the elongated hollow tube, adjacent the distal end thereof. The auxiliary sterilizing tube includes a closed distal end and an open proximal end. The closed distal end is secured to the endotracheal tube, adjacent the inflatable cuff. A plurality of pores are formed through at least a portion of the sterilizing tube. The sterilizing agent source can be an infusion syringe or the like. The sterilizing agent source is connected to the open proximal end of the sterilizing tube for injecting a sterilizing agent, such as chlorhexidine, into the sterilizing tube, which is distributed to the elongated hollow tube through the pores.

The auxiliary sterilizing tube is preferably fixed around the endotracheal tube in a helical manner. For example, ten separate coils of the helically wound auxiliary sterilizing tube may be formed about the endotracheal tube, although it should be understood that the number of coils formed may be varied dependent upon the desired flow rate of the sterilizing agent. The number of pores preferably ranges between 14 and 16, with the average spacing therebetween being approximately 26 mm to approximately 34 mm along the auxiliary sterilizing tube. It should be understood that variation in spacing of the pores may be based on the pressure distribution after the auxiliary sterilizing tube is helically wound about the endotracheal tube. The size of the pores is preferably 30 G for proximally positioned pores and 29 G for distally positioned holes to maintain uniform flow rate of the sterilizing agent. For a conventional endotracheal tube, the pores are formed beginning below the level marked on the endotracheal tube. Preferably, for use with a conventional endotracheal tube, the pores begin at approximately 20 mm beyond the mouth fixation portion; i.e. at a level of approximately 20 cm, as marked on the endotracheal tube. The pores end at a level of approximately 20 mm above the inflatable cuff. The infusion flow rate is regulated to enable 2.5 mL/hour. An exemplary pressure used for these exemplary values is 5-6 mm Hg.

These and other features of the present invention will become readily apparent upon further review of the following specification and drawings.

### BRIEF DESCRIPTION OF DRAWINGS

The sole drawing FIGURE is a perspective view of an endotracheal tube with an auxiliary sterilizing tube according to the present invention.

Similar reference characters denote corresponding features consistently throughout the attached drawings.

### BEST MODES FOR CARRYING OUT THE INVENTION

The endotracheal tube system 10 includes an endotracheal tube 12, an auxiliary sterilizing tube 20, and a sterilizing agent source 28. The sterilizing agent source 28 provides a sterilizing agent to the endotracheal tube 12 through the auxiliary sterilizing tube 10 for sterilization of the endotracheal tube 12 while it is in use. As is conventionally known, and as shown in the sole drawing FIGURE, the endotracheal tube 12 can be an elongated hollow tube having a distal end 14 and a proximal end 16, with the distal end 14 being adapted for insertion into a trachea of a patient. An inflatable cuff 18 is disposed on the elongated hollow tube, adjacent the distal end 14. It should be understood that endotracheal tube 12 may be any conventional type of endotracheal tube, such as, for example, the Mallinckrodt Cuffed Basic Endotracheal Tube manufactured by Medtronic® of Minneapolis, Minn. Such an exemplary endotracheal tube may have an exemplary internal diameter of 8.0 mm, an exemplary outer diameter of 11.8 mm, and be cuffed for subglottic suction, as shown in the example of the sole drawing FIGURE.

The auxiliary sterilizing tube 20 has a closed, distal end 24 and an open, proximal end 22. The closed, distal end 24 of the sterilizing tube 20 is secured to the elongated hollow tube 12 adjacent the inflatable cuff 18, as shown. A plurality of pores 26 are formed through at least a portion of the sterilizing tube 20, preferably adjacent the closed, distal end 24, as shown in the example of the sole drawing FIGURE.

As shown in the sole drawing FIGURE, the auxiliary sterilizing tube 20 is preferably fixed around the endotracheal tube 12 in a helical manner. For example, ten separate coils of the helically wound auxiliary sterilizing tube 20 may be formed about the endotracheal tube 12, although it should be understood that the number of coils formed may be varied dependent upon the desired flow rate of the sterilizing agent. The number of pores 26 preferably ranges between 14 and 16, with the average spacing therebetween being approximately 26 mm to approximately 34 mm along the auxiliary sterilizing tube 20. It should be understood that variation in spacing of the pores 26 may be based on the pressure distribution after the auxiliary sterilizing tube 20 is helically wound about the endotracheal tube 12. The size of the pores 26 is preferably 30 G for the proximally positioned pores and 29 G for distally positioned holes to maintain uniform flow rate of the sterilizing agent. For a conventional endotracheal tube, for example, the Mallinckrodt Cuffed Basic Endotracheal Tube manufactured by Medtronic® of Minneapolis, Minn., the pores 26 are formed beginning at approximately 20 mm beyond the mouth fixation portion (indicated as “M” in the sole drawing FIGURE); i.e. at a level of approximately 20 cm, as marked on the endotracheal tube 12. The pores 26 end at a level of approximately 20 mm above the inflatable cuff 18. The infusion flow rate is regulated to enable 2.5 mL/hour. An exemplary pressure used for these exemplary values is 5-6 mm Hg.

The sterilizing agent source 28 can include any suitable type of dispenser for injecting a sterilizing agent into the sterilizing tube 20. Preferably, the sterilizing agent is chlorhexidine. In the example of the sole drawing FIGURE, the sterilizing agent source 28 is a conventional infusion syringe. It should be understood, however, that any suitable type of pump, infusion pump, syringe pump or the like may be used for controlled injection of the chlorhexidine into auxiliary sterilizing tube 20. The infusion syringe 28 may be directly connected to the open, proximal end 22 of the sterilizing tube 20 for injecting chlorhexidine into the sterilizing tube 20 or, as shown, a conventional three-way stopcock 30 or the like may be interposed therebetween, allowing for tertiary connection to an external suction source for suction of oral secretions. Injection of the chlorhexidine into the sterilizing tube 20 provides for distribution thereof to the elongated hollow tube 12 through the pores 26.

For the exemplary type of endotracheal tube 12 described above, the auxiliary sterilizing tube 20 may have a corresponding exemplary length of 70 cm and an exemplary diameter of 2.5 mm, and may be manufactured from polyvinyl chloride or any other suitable type of material which is fluid impermeable and non-hazardous to the patient. In the sole drawing FIGURE, the open, proximal end 22 of sterilizing tube 20 is shown having a conventional connector for connection with a conventional infusion syringe (typically a wide end adapted for connection with a 50 cm syringe end), although it should be understood that any suitable type of connector may be utilized, dependent upon the particular type of syringe 28 or the like and/or the particular type of three-way stopcock 30 for possible drainage of accumulated mouth secretions.

For the above exemplary dimensions, a distal end portion of the sterilizing tube 20, e.g., the last 16 cm of the sterilizing tube 20, adjacent the closed distal end 24, includes pores 26, with the remainder of the sterilizing tube 20 being continuous and impermeable. The pores each have a diameter of approximately 0.2 mm. The chlorhexidine may be injected at a rate of 60 mL per 24 hours of intubation, distributed every six hours, similar to the dosage conventionally used for chlorhexidine sterilizing of a patient's mouth to prevent VAP.

It is to be understood that the present invention is not limited to the embodiments described above, but encompasses any and all embodiments within the scope of the following claims.

I claim:

1. An endotracheal tube system, comprising:
an elongated hollow tube having a distal end and a proximal end, the distal end being adapted for insertion into a trachea of a patient;
an inflatable cuff disposed on said elongated hollow tube adjacent the distal end thereof;
a sterilizing tube having a closed distal end and an open proximal end, the closed distal end being secured to the elongated hollow tube adjacent the inflatable cuff, a plurality of pores being formed through at least a portion of the sterilizing tube; and
a sterilizing agent source for distributing a sterilizing agent to the elongated hollow tube through the pores of the sterilizing tube.

2. The endotracheal tube system as recited in claim 1, wherein said sterilizing agent source comprises an infusion syringe and said sterilizing agent comprises chlorhexidine.

3. The endotracheal tube system as recited in claim 2, further comprising a three-way stopcock for providing selective fluid communication between the sterilizing tube, the infusion syringe and an external suction source.

4. The endotracheal tube system as recited in claim 1, wherein the sterilizing tube is helically wound about the elongated hollow tube.

5. An endotracheal tube system, comprising:
an elongated hollow tube having a distal end and a proximal end, the distal end being adapted for insertion into a trachea of a patient;
an inflatable cuff disposed on said elongated hollow tube adjacent the distal end thereof;
a sterilizing tube having a closed distal end and an open proximal end, the closed distal end being secured to the elongated hollow tube adjacent the inflatable cuff, a plurality of pores being formed through at least a portion of the sterilizing tube; and
an infusion syringe for injecting chlorhexidine into the sterilizing tube, through the open proximal end thereof, for distribution of the chlorhexidine to the elongated hollow tube through the pores formed through the sterilizing tube.

6. The endotracheal tube system as recited in claim 5, further comprising a three-way stopcock for providing selective fluid communication between the sterilizing tube, the infusion syringe and an external suction source.

7. The endotracheal tube system as recited in claim 5, wherein the sterilizing tube is helically wound about the elongated hollow tube.

8. An endotracheal tube system, comprising:
an elongated hollow tube having a distal end and a proximal end, the distal end being adapted for insertion into a trachea of a patient;
an inflatable cuff disposed on said elongated hollow tube adjacent the distal end thereof; and
a sterilizing tube having a closed distal end and an open proximal end, the closed distal end being secured to the elongated hollow tube adjacent the inflatable cuff, a plurality of pores being formed through at least a portion of the sterilizing tube.

9. The endotracheal tube as recited in claim 8, further comprising an infusion syringe for injecting chlorhexidine into the sterilizing tube, through the open proximal end thereof, for distribution of the chlorhexidine to the elongated hollow tube through the pores formed through the sterilizing tube.

10. The endotracheal tube as recited in claim 9, further comprising a three-way stopcock for providing selective fluid communication between the sterilizing tube, the infusion syringe and an external suction source.

11. The endotracheal tube system as recited in claim 8, wherein the sterilizing tube is helically wound about the elongated hollow tube.
